(19) United States
(12) Patent Application Publication
(10) Pub. No.: US 2025/0059507 A1
(43) Pub. Date: Feb. 20, 2025

(54) Generation of Tolerance Promoting CAR-T Cells by Enhancement of NR2F6

(71) Applicant: Regen Biopharma, Inc., La Mesa, CA (US)

(72) Inventors: Thomas Ichim, San Diego, CA (US); David Koos, Le Mesa, CA (US)

(73) Assignee: Regen Biopharma, Inc., La Mesa, CA (US)

(21) Appl. No.: 18/804,630

(22) Filed: Aug. 14, 2024

Related U.S. Application Data
(60) Provisional application No. 63/520,062, filed on Aug. 16, 2023.

(51) Int. Cl. C12N 5/0783

(52) U.S. Cl. CPC C12N 5/0636; C12N 2510/00

(57) ABSTRACT

Compositions of matter and methods of treatment based on generation of T cells capable of ameliorating or treating pathologies associated with improper immune activation. Chimeric antigen receptor (CAR) T cells are generated which possess tolerogenic properties based on enhanced expression of NR2F6. NR2F6 is enhanced through transfection of cells with a modified siRNA sequence. NR2F6 expression is upregulated through tissue culture modification, and NR2F6 expression vectors are introduced to the cell of interest. Methods and compositions to treat autoimmunity, transplant rejection, and conditions associated with pathological immune activation.

### CROSS-REFERENCE TO RELATED APPLICATIONS

This application claims priority to U.S. Provisional Application No. 63/520,062, filed Aug. 16, 2023, and titled “Generation of Tolerance Promoting CAR-T Cells by Enhancement of NR2F6”, which is hereby incorporated by reference in its entirety.

### FIELD OF THE INVENTION

The teachings herein relate to the field of immunotherapy, more specifically, the invention belongs to the field of prevention and/or reversing autoimmune disease or organ transplant rejection.

### BACKGROUND OF THE INVENTION

Immunotherapy has resulted in treatment of numerous cancer, in many cases with reduced toxicity as compared to conventional chemotherapy or radiation approaches. In the case of cancer the goal of immunotherapy is breaking of self tolerance to tumor antigens. In the context of autoimmunity, the goal of immunotherapy is creation or re-establishment of immunological tolerance. Despite previous attempts to induce tolerance using oral antigen deliver, or solubilized intravenous administration, success in Phase III trials has not materialized. The current invention provides means of inducing tolerance using a T cells which overexpress NR2F6.

### SUMMARY OF THE INVENTION

Preferred embodiments are directed to tolerance promoting T cell generated through augmentation of NR2F6 levels in a T cell, T cell progenitor, or pluripotent stem cell giving rise to a T cell.

Preferred tolerance promoting T cells include embodiments wherein augmentation of NR2F6 is achieved through introduction of one or more short hairpin RNA molecules targeting NR2F6.

Preferred tolerance promoting T cells include embodiments wherein said introduction of one or more short hairpin RNA molecules targeting NR2F6 results in upregulation of NR2F6 activity.

Preferred tolerance promoting T cells include embodiments wherein said introduction of one or more short hairpin RNA molecules targeting NR2F6 results in upregulation of NR2F6 transcription.

Preferred tolerance promoting T cells include embodiments wherein said introduction of one or more short hairpin RNA molecules targeting NR2F6 results in upregulation of NR2F6 translation.

Preferred tolerance promoting T cells include embodiments wherein said introduction of one or more short hairpin RNA molecules targeting NR2F6 results in upregulation of NR2F6 protein half-life.

Preferred tolerance promoting T cells include embodiments wherein augmentation of NR2F6 is achieved through introduction of one or more short interfering RNA molecules targeting NR2F6.

Preferred tolerance promoting T cells include embodiments wherein said introduction of one or more short interfering RNA molecules targeting NR2F6 results in upregulation of NR2F6 activity.

Preferred tolerance promoting T cells include embodiments wherein said introduction of one or more short interfering RNA molecules targeting NR2F6 results in upregulation of NR2F6 transcription.

Preferred tolerance promoting T cells include embodiments wherein said introduction of one or more short interfering RNA molecules targeting NR2F6 results in upregulation of NR2F6 translation.

Preferred tolerance promoting T cells include embodiments wherein said introduction of one or more short interfering RNA molecules targeting NR2F6 results in upregulation of NR2F6 protein half-life.

Preferred tolerance promoting T cells include embodiments wherein said cell is induced to upregulate expression of NR2F6 by transfection of a gene construct encoding said NR2F6 or a homologue thereof.

Preferred tolerance promoting T cells include embodiments wherein said gene construct encoding said NR2F6 or a homologue thereof is transfected by means of lipid-based systems.

Preferred tolerance promoting T cells include embodiments wherein said gene construct encoding said NR2F6 or a homologue thereof is transfected by means of viral-based systems.

Preferred tolerance promoting T cells include embodiments wherein said gene construct encoding said NR2F6 or a homologue thereof is transfected by means of adenoviral-based systems.

Preferred tolerance promoting T cells include embodiments wherein said gene construct encoding said NR2F6 or a homologue thereof is transfected by means of lentiviral-based systems.

Preferred tolerance promoting T cells include embodiments wherein said gene construct encoding said NR2F6 or a homologue thereof is transfected by means of adeno associated viral-based systems.

Preferred tolerance promoting T cells include embodiments wherein said gene construct encoding said NR2F6 or a homologue thereof is gene edited into said T cell, T cell progenitor, or stem cell to be differentiated into said T cell.

Preferred tolerance promoting T cells include embodiments wherein said NR2F6 is upregulated by means of culture conditions.

Preferred tolerance promoting T cells include embodiments wherein said culture condition is treatment with a histone deacetylase inhibitor.

Preferred tolerance promoting T cells include embodiments wherein said T cell is a CD4 T cell.

Preferred tolerance promoting T cells include embodiments wherein said T cell is a CD8 T cell.

Preferred tolerance promoting T cells include embodiments wherein said T cell is a chimeric antigen receptor (CAR) T cell.

Preferred tolerance promoting T cells include embodiments wherein said chimeric antigen receptor recognizes an autoantigen.

Preferred tolerance promoting T cells include embodiments wherein said autoantigen is GAD65.

Preferred tolerance promoting T cells include embodiments wherein said autoantigen is insulin.

Preferred tolerance promoting T cells include embodiments wherein said autoantigen is insulin beta chain.

Preferred tolerance promoting T cells include embodiments wherein said autoantigen is IA2.

Preferred tolerance promoting T cells include embodiments wherein said autoantigen is phogrin.

Preferred tolerance promoting T cells include embodiments wherein said autoantigen is hsp90b.

Preferred tolerance promoting T cells include embodiments wherein said autoantigen is carboxypeptidase E.

Preferred tolerance promoting T cells include embodiments wherein said autoantigen is islet glucose 6 phosphatase-related protein.

Preferred tolerance promoting T cells include embodiments wherein said autoantigen is islet amyloid polypeptide.

Preferred tolerance promoting T cells include embodiments wherein said autoantigen is Reg3a.

Preferred tolerance promoting T cells include embodiments wherein said autoantigen is ICA69.

Preferred tolerance promoting T cells include embodiments wherein said autoantigen is imogen 38.

Preferred tolerance promoting T cells include embodiments wherein said autoantigen is peripherin.

Preferred tolerance promoting T cells include embodiments wherein said autoantigen is sox13.

Preferred tolerance promoting T cells include embodiments wherein said autoantigen is GAD67.

Preferred tolerance promoting T cells include embodiments wherein said autoantigen is Hsp65.

Preferred tolerance promoting T cells include embodiments wherein said autoantigen is DnaJ.

Preferred tolerance promoting T cells include embodiments wherein said autoantigen is DnaJ.

Preferred tolerance promoting T cells include embodiments wherein said autoantigen is immunoglobulin binding protein.

Preferred tolerance promoting T cells include embodiments wherein said autoantigen is heterogeneous nuclear ribonucleoprotein A2.

Preferred tolerance promoting T cells include embodiments wherein said autoantigen is calpastatin.

Preferred tolerance promoting T cells include embodiments wherein said autoantigen is type II collagen.

Preferred tolerance promoting T cells include embodiments wherein said autoantigen is glucose-6-phosphate isomerase.

Preferred tolerance promoting T cells include embodiments wherein said autoantigen is gp39.

Preferred tolerance promoting T cells include embodiments wherein said autoantigen is mannose binding lectin.

Preferred tolerance promoting T cells include embodiments wherein said autoantigen is citrullinated vimentin.

Preferred tolerance promoting T cells include embodiments wherein said autoantigen is fibrinogen.

Preferred tolerance promoting T cells include embodiments wherein said autoantigen is alpha enolase.

Preferred tolerance promoting T cells include embodiments wherein said autoantigen is myelin basic protein.

Preferred tolerance promoting T cells include embodiments wherein said autoantigen is anoctamin-2.

Preferred tolerance promoting T cells include embodiments wherein said autoantigen is myelin oligodendrocyte glycoprotein.

Preferred tolerance promoting T cells include embodiments wherein said autoantigen is KIR4.1.

Preferred tolerance promoting T cells include embodiments wherein said autoantigen is aquaporin-4.

Preferred tolerance promoting T cells include embodiments wherein said autoantigen is CRYAB.

Preferred tolerance promoting T cells include embodiments wherein said autoantigen is PLP.

Preferred tolerance promoting T cells include embodiments wherein said autoantigen is contactin-2.

Preferred tolerance promoting T cells include embodiments wherein said T cell is transfected with FoxP3.

Preferred tolerance promoting T cells include embodiments wherein said T cell expresses CTLA4.

Preferred tolerance promoting T cells include embodiments wherein said T cell expresses TGF-beta.

Preferred tolerance promoting T cells include embodiments wherein said T cell expresses LAG3.

Preferred tolerance promoting T cells include embodiments wherein said T cell expresses TIM3.

Preferred tolerance promoting T cells include embodiments wherein said T cell secretes IL-10 after CD3 ligation.

Preferred tolerance promoting T cells include embodiments wherein said T cell secretes VEGF after CD3 ligation.

Preferred tolerance promoting T cells include embodiments wherein said T cell secretes PDGF-BB after CD3 ligation.

Preferred tolerance promoting T cells include embodiments wherein said T cell secretes soluble HLA-G after CD3 ligation.

Preferred tolerance promoting T cells include embodiments wherein said T cell secretes interleukin-1 receptor antagonist after CD3 ligation.

Preferred tolerance promoting T cells include embodiments wherein said T cell inhibits maturation of dendritic cells.

Preferred tolerance promoting T cells include embodiments wherein said dendritic cell maturation is upregulation of IL-12 production.

Preferred tolerance promoting T cells include embodiments wherein said dendritic cell maturation is upregulation of IL-15 production.

Preferred tolerance promoting T cells include embodiments wherein said dendritic cell maturation is upregulation of IL-18 production.

Preferred tolerance promoting T cells include embodiments wherein said dendritic cell maturation is upregulation of IL-17 production.

Preferred tolerance promoting T cells include embodiments wherein said dendritic cell maturation is upregulation of IL-21 production.

Preferred tolerance promoting T cells include embodiments wherein said dendritic cell maturation is upregulation of IL-23 production.

Preferred tolerance promoting T cells include embodiments wherein said dendritic cell maturation is upregulation of IL-27 production.

Preferred tolerance promoting T cells include embodiments wherein said dendritic cell maturation is upregulation of IL-33 production.

Preferred tolerance promoting T cells include embodiments wherein said dendritic cell maturation is upregulation of IL-1 beta production.

Preferred tolerance promoting T cells include embodiments wherein said dendritic cell maturation is upregulation of HMGB1 production.

Preferred tolerance promoting T cells include embodiments wherein said dendritic cell maturation is upregulation of interferon alpha production.

Preferred tolerance promoting T cells include embodiments wherein said dendritic cell maturation is upregulation of interferon gamma production.

Preferred tolerance promoting T cells include embodiments wherein said dendritic cell maturation is upregulation of lymphotoxin production.

Preferred tolerance promoting T cells include embodiments wherein said dendritic cell maturation is upregulation of TNF-alpha production.

Preferred tolerance promoting T cells include embodiments wherein said dendritic cell maturation is upregulation of MIP-1 alpha production.

Preferred tolerance promoting T cells include embodiments wherein said dendritic cell maturation is upregulation of MIP-1 beta production.

Preferred tolerance promoting T cells include embodiments wherein said dendritic cell maturation is upregulation of RANTES production.

Preferred tolerance promoting T cells include embodiments wherein said dendritic cell maturation is upregulation of TRANCE production.

Preferred tolerance promoting T cells include embodiments wherein said dendritic cell maturation is upregulation of MCP production.

Preferred tolerance promoting T cells include embodiments wherein said dendritic cell maturation is increased expression of CD40.

Preferred tolerance promoting T cells include embodiments wherein said dendritic cell maturation is increased expression of CD80.

Preferred tolerance promoting T cells include embodiments wherein said dendritic cell maturation is increased expression of CD86.

Preferred tolerance promoting T cells include embodiments wherein said dendritic cell maturation is increased expression of LFA-1.

Preferred tolerance promoting T cells include embodiments wherein said dendritic cell maturation is increased expression of ICAM-1.

Preferred tolerance promoting T cells include embodiments wherein said dendritic cell maturation is increased expression of transporter associated protein-1.

Preferred tolerance promoting T cells include embodiments wherein said dendritic cell maturation is increased antigen presentation activity.

Preferred tolerance promoting T cells include embodiments wherein said dendritic cell maturation is decreased phagocytic activity.

Preferred tolerance promoting T cells include embodiments wherein said T cell is capable of suppressing proliferation of other T cells.

Preferred tolerance promoting T cells include embodiments wherein said T cell proliferation is stimulated by a mitogen, wherein said mitogen is selected from a group comprising of: a) a lectin; b) concanavalin-A; c) phytohemagglutinin; d) pokeweed mitogen; e) anti-CD3 antibody; f) anti-CD28 antibody and g) interleukin-2.

Preferred embodiments are directed to methods of creating T regulatory cells, said method comprising augmentation of NR2F6 expression in a T cell.

Preferred methods include embodiments wherein said T regulatory cells are autologous.

Preferred methods include embodiments wherein said T regulatory cells are allogeneic.

Preferred methods include embodiments wherein said T regulatory cells are xenogeneic.

Preferred methods include embodiments wherein said T regulatory cells are expanded from differentiated or semi-differentiated sources.

Preferred methods include embodiments wherein said differentiated or semi-differentiated source is a peripheral blood.

Preferred methods include embodiments wherein said peripheral blood is extracted subsequent to administration of one or more agents capable of increasing number of T regulatory cells in said peripheral blood.

Preferred methods include embodiments wherein said agent that increases number of T regulatory cells is G-CSF.

Preferred methods include embodiments wherein said agent that increases number of T regulatory cells is GM-CSF.

Preferred methods include embodiments wherein said agent that increases number of T regulatory cells is M-CSF.

Preferred methods include embodiments wherein said agent that increases number of T regulatory cells is FLT-3L.

Preferred methods include embodiments wherein said differentiated or semi-differentiated source is cord blood.

Preferred methods include embodiments wherein said differentiated or semi-differentiated source is menstrual blood.

Preferred methods include embodiments wherein said differentiated or semi-differentiated source is bone marrow.

Preferred methods include embodiments wherein said differentiated or semi-differentiated source is adipose tissue.

Preferred methods include embodiments wherein said differentiated or semi-differentiated source is placental tissue.

Preferred methods include embodiments wherein said differentiated or semi-differentiated source is fallopian tube tissue.

Preferred methods include embodiments wherein said differentiated or semi-differentiated source is omental tissue.

Preferred methods include embodiments wherein said differentiated or semi-differentiated source is tonsillar tissue.

Preferred methods include embodiments wherein said differentiated or semi-differentiated source is umbilical cord tissue.

Preferred methods include embodiments wherein said differentiated or semi-differentiated source is Wharton's Jelly tissue.

Preferred methods include embodiments wherein said differentiated or semi-differentiated source is subdermal tissue.

Preferred methods include embodiments wherein said differentiated or semi-differentiated source is subintestinal submucosal tissue.

Preferred methods include embodiments wherein said differentiated or semi-differentiated source is endometrial tissue.

Preferred methods include embodiments wherein said T regulatory cells are expanded from undifferentiated sources.

Preferred methods include embodiments wherein said undifferentiated source is a pluripotent stem cell.

Preferred methods include embodiments wherein said undifferentiated source is a multipotent stem cell.

Preferred methods include embodiments wherein said undifferentiated source is a committed progenitor cell.

Preferred methods include embodiments wherein said pluripotent stem cell is a totipotent stem cell.

Preferred methods include embodiments wherein said pluripotent stem cell expresses SSEA4.

Preferred methods include embodiments wherein said pluripotent stem cell expresses OCT4.

Preferred methods include embodiments wherein said pluripotent stem cell expresses NANOG.

Preferred methods include embodiments wherein said pluripotent stem cell expresses PIM1.

Preferred methods include embodiments wherein said pluripotent stem cell expresses KLF4.

Preferred methods include embodiments wherein said pluripotent stem cell expresses SOX2.

Preferred methods include embodiments wherein said pluripotent stem cell expresses wnt.

Preferred methods include embodiments wherein said pluripotent stem cell expresses JAGGED.

Preferred methods include embodiments wherein said pluripotent stem cell expresses hTERT.

Preferred methods include embodiments wherein said pluripotent stem cell is an inducible pluripotent stem cell.

Preferred methods include embodiments wherein said pluripotent stem cell is a somatic cell nuclear transfer derived stem cell.

Preferred methods include embodiments wherein said pluripotent stem cell is a parthenogenesis derived stem cell.

Preferred methods include embodiments wherein said pluripotent stem cell is an inducible pluripotent stem cell.

Preferred methods include embodiments wherein said inducible pluripotent stem cell is expanded on fetal fibroblasts.

Preferred methods include embodiments wherein said inducible pluripotent stem cell stem cell is expanded on thymic medullary epithelial cells.

Preferred methods include embodiments wherein said inducible pluripotent stem cell stem cell is expanded on immortalized thymic medullary epithelial cells.

Preferred methods include embodiments wherein said inducible pluripotent stem cell is expanded on mesenchymal stem cells.

Preferred methods include embodiments wherein said mesenchymal stem cells express STRO-1.

Preferred methods include embodiments wherein said mesenchymal stem cells express CD90.

Preferred methods include embodiments wherein said mesenchymal stem cells express CD105.

Preferred methods include embodiments wherein said mesenchymal stem cells express CD132.

Preferred methods include embodiments wherein said mesenchymal stem cells do not express CD14

Preferred methods include embodiments wherein said mesenchymal stem cells do not express CD16

Preferred methods include embodiments wherein said mesenchymal stem cells do not express CD45

Preferred methods include embodiments wherein said mesenchymal stem cells do not express IL-12 receptor.

Preferred methods include embodiments wherein said mesenchymal stem cells are derived from pluripotent stem cells.

Preferred methods include embodiments wherein said mesenchymal stem cells are derived from bone marrow stem cells.

Preferred methods include embodiments wherein said bone marrow stem cells are cultured under hypoxic conditions.

Preferred methods include embodiments wherein said bone marrow stem cells express CD133.

Preferred methods include embodiments wherein said bone marrow stem cells express CD73.

Preferred methods include embodiments wherein said T regulatory cells are expanded by culture in a liquid media.

Preferred methods include embodiments wherein said media is alpha MEM.

Preferred methods include embodiments wherein said media is Eagles Media.

Preferred methods include embodiments wherein said media is RPMI-1640.

Preferred methods include embodiments wherein said media is AIM-V.

Preferred methods include embodiments wherein said media is DMEM.

Preferred methods include embodiments wherein said media is Opti-MEM.

Preferred methods include embodiments wherein said media is Iscove's Media.

Preferred methods include embodiments wherein said media contains conditioned media.

Preferred methods include embodiments wherein said conditioned media is media that has been exposed to activated leukocytes.

Preferred methods include embodiments wherein said activated leukocytes are T cells.

Preferred methods include embodiments wherein said T cells are CD3 expressing T cells.

Preferred methods include embodiments wherein said T cells are CD4 expressing T cells.

Preferred methods include embodiments wherein said T cells are CD8 expressing T cells.

Preferred methods include embodiments wherein said T cells are CD28 expressing T cells.

Preferred methods include embodiments wherein leukocytes are substantially enriched for mononuclear cells.

Preferred methods include embodiments wherein said enrichment is performed based on density.

Preferred methods include embodiments wherein said enrichment is performed using Ficoll and/or Percoll density gradients.

Preferred methods include embodiments wherein leukocytes are activated by culture with an allogeneic population of leukocytes.

Preferred methods include embodiments wherein leukocytes are activated by culture with an allogeneic population of antigen presenting cells.

Preferred methods include embodiments wherein said antigen presenting cells are B cells.

Preferred methods include embodiments wherein said B cells express CD5.

Preferred methods include embodiments wherein said B cells express CD10.

Preferred methods include embodiments wherein said B cells express CD19.

Preferred methods include embodiments wherein said B cells express CD20.

Preferred methods include embodiments wherein said B cells express IL-4 receptor.

Preferred methods include embodiments wherein said B cells express IL-13 receptor.

Preferred methods include embodiments wherein said B cells express IL-6 receptor.

Preferred methods include embodiments wherein said antigen presenting cells are fibroblasts.

Preferred methods include embodiments wherein said antigen presenting cells are fibroblasts treated with interferon gamma.

Preferred methods include embodiments wherein said antigen presenting cells are monocytes.

Preferred methods include embodiments wherein said antigen presenting cells are monocytes treated with interferon gamma.

Preferred methods include embodiments wherein said antigen presenting cells are macrophages.

Preferred methods include embodiments wherein said antigen presenting cells are macrophages treated with interferon gamma.

Preferred methods include embodiments wherein said antigen presenting cells are dendritic cells.

Preferred methods include embodiments wherein said antigen presenting cells are myeloid dendritic cells.

Preferred methods include embodiments wherein said antigen presenting cells are lymphoid dendritic cells.

Preferred methods include embodiments wherein said antigen presenting cells are neutrophils.

Preferred methods include embodiments wherein said antigen presenting cells are N1 neutrophils.

Preferred methods include embodiments wherein said antigen presenting cells are endothelial cells.

Preferred methods include embodiments wherein said antigen presenting cells are endothelial cells treated with interferon gamma.

Preferred methods include embodiments wherein said conditioned media is media that has been exposed to mesenchymal stem cells.

### DETAILED DESCRIPTION OF THE INVENTION

The invention provides means of utilizing T cells as a tolerogenic cell through the upregulation of NR2F6 expression. In one embodiment the invention discloses the use of RNA interference inducing techniques to paradoxically elicit upregulation of NR2F6 expression. In other embodiments the invention provides means of gene engineering T cells to increase NR2F6 expression. Application to CAR-T cells, polyclonal T cells and clonal T cells is envisioned within the current invention.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “agent”, “ligand”, or “agent that binds a cell surface moiety”, as used herein, refers to a molecule that binds to a defined population of cells. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), a carbohydrate, or the like. Within the specification and in the context of T cell stimulation, antibodies and natural ligands are used as prototypical examples of such agents.

The terms “agent that binds a cell surface moiety” and “cell surface moiety”, as used herein, are used in the context of a ligand/anti-ligand pair. Accordingly, these molecules should be viewed as a complementary/anti-complementary set of molecules that demonstrate specific binding, generally of relatively high affinity.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

The term “expression” as used herein is defined as the transcription and/or translation of a particular nucleotide sequence driven by its promoter.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Self-protein,” “self-polypeptide,” or self-peptide” are used herein interchangeably and refer to any protein, polypeptide, or peptide, or fragment or derivative thereof that: is encoded within the genome of the animal; is produced or generated in the animal; may be modified posttranslationally at some time during the life of the animal; and, is present in the animal non-physiologically. The term “non-physiological” or “non-physiologically” when used to describe the self-protein(s), -polypeptide(s), or -peptide(s) of this invention means a departure or deviation from the normal role or process in the animal for that self-protein, -polypeptide, or -peptide. When referring to the self-protein, -polypeptide or -peptide as “associated with a disease” or “involved in a disease” it is understood to mean that the self-protein, -polypeptide, or -peptide may be modified in form or structure and thus be unable to perform its physiological role or process or may be involved in the pathophysiology of the condition or disease either by inducing the pathophysiology; mediating or facilitating a pathophysiologic process; and/or by being the target of a pathophysiologic process. For example, in autoimmune disease, the immune system aberrantly attacks self-proteins causing damage and dysfunction of cells and tissues in which the self-protein is expressed and/or present. Alternatively, the self-protein, -polypeptide or -peptide can itself be expressed at non-physiological levels and/or function non-physiologically. For example in neurodegenerative diseases self-proteins are aberrantly expressed, and aggregate in lesions in the brain thereby causing neural dysfunction. In other cases, the self-protein aggravates an undesired condition or process. For example in osteoarthritis, self-proteins including collagenases and matrix metalloproteinases aberrantly degrade cartilage covering the articular surface of joints. Examples of posttranslational modifications of self-protein(s), -polypeptide(s) or -peptide(s) are glycosylation, addition of lipid groups, reversible phosphorylation, addition of dimethylarginine residues, citrullination, and proteolysis, and more specifically citrullination of fillagrin and fibrin by peptidyl arginine deaminase (PAD), alpha β-crystallin phosphorylation, citrullination of MBP, and SLE autoantigen proteolysis by caspases and granzymes. Immunologically, self-protein, -polypeptide or -peptide would all be considered host self-antigens and under normal physiological conditions are ignored by the host immune system through the elimination, inactivation, or lack of activation of immune cells that have the capacity to recognize self-antigens through a process designated “immune tolerance.” A self-protein, -polypeptide, or -peptide does not include immune proteins, polypeptides, or peptides which are molecules expressed physiologically exclusively by cells of the immune system for the purpose of regulating immune function. The immune system is the defense mechanism that provides the means to make rapid, highly specific, and protective responses against the myriad of potentially pathogenic microorganisms inhabiting the animal's world. Examples of immune protein(s), polypeptide(s) or peptide(s) are proteins comprising the T-cell receptor, immunoglobulins, cytokines including the type I interleukins, and the type II cytokines, including the interferons and IL-10, TNF, lymphotoxin, and the chemokines such as macrophage inflammatory protein-1 alpha and beta, monocyte-chemotactic protein and RANTES, and other molecules directly involved in immune function such as Fas-ligand. There are certain immune protein(s), polypeptide(s) or peptide(s) that are included in the self-protein, -polypeptide or -peptide of the invention and they are: class I MHC membrane glycoproteins, class II MHC glycoproteins and osteopontin. Self-protein, -polypeptide or -peptide does not include proteins, polypeptides, and peptides that are absent from the subject, either entirely or substantially, due to a genetic or acquired deficiency causing a metabolic or functional disorder, and are replaced either by administration of said protein, polypeptide, or peptide or by administration of a polynucleotide encoding said protein, polypeptide or peptide (gene therapy). Examples of such disorders include Duchenne' muscular dystrophy, Becker's muscular dystrophy, cystic fibrosis, phenylketonuria, galactosemia, maple syrup urine disease, and homocystinuria. Self-protein, -polypeptide or -peptide does not include proteins, polypeptides, and peptides expressed specifically and exclusively by cells which have characteristics that distinguish them from their normal counterparts, including: (1) clonality, representing proliferation of a single cell with a genetic alteration to form a clone of malignant cells, (2) autonomy, indicating that growth is not properly regulated, and (3) anaplasia, or the lack of normal coordinated cell differentiation. Cells have one or more of the foregoing three criteria are referred to either as neoplastic, cancer or malignant cells.

“Modulation of,” “modulating”, or “altering an immune response” as used herein refers to any alteration of an existing or potential immune responses against self-molecules, including, e.g., nucleic acids, lipids, phospholipids, carbohydrates, self-polypeptides, protein complexes, or ribonucleoprotein complexes, that occurs as a result of administration of a polynucleotide encoding a self-polypeptide. Such modulation includes any alteration in presence, capacity, or function of any immune cell involved in or capable of being involved in an immune response. Immune cells include B cells, T cells, NK cells, NK T cells, professional antigen-presenting cells, non-professional antigen-presenting cells, inflammatory cells, or any other cell capable of being involved in or influencing an immune response. “Modulation” includes any change imparted on an existing immune response, a developing immune response, a potential immune response, or the capacity to induce, regulate, influence, or respond to an immune response. Modulation includes any alteration in the expression and/or function of genes, proteins and/or other molecules in immune cells as part of an immune response.

“Modulation of an immune response” includes, for example, the following: elimination, deletion, or sequestration of immune cells; induction or generation of immune cells that can modulate the functional capacity of other cells such as autoreactive lymphocytes, antigen presenting cells (APCs), or inflamatory cells; induction of an unresponsive state in immune cells (i.e., anergy); increasing, decreasing, or changing the activity or function of immune cells or the capacity to do so, including but not limited to altering the pattern of proteins expressed by these cells. Examples include altered production and/or secretion of certain classes of molecules such as cytokines, chemokines, growth factors, transcription factors, kinases, costimulatory molecules, or other cell surface receptors; or any combination of these modulatory events.

For example, a polynucleotide encoding a self-polypeptide can modulate an immune response by eliminating, sequestering, or inactivating immune cells mediating or capable of mediating an undesired immune response; inducing, generating, or turning on immune cells that mediate or are capable of mediating a protective immune response; changing the physical or functional properties of immune cells; or a combination of these effects. Examples of measurements of the modulation of an immune response include, but are not limited to, examination of the presence or absence of immune cell populations (using flow cytometry, immunohistochemistry, histology, electron microscopy, polymerase chain reaction (PCR)); measurement of the functional capacity of immune cells including ability or resistance to proliferate or divide in response to a signal (such as using T cell proliferation assays and pepscan analysis based on 3H-thymidine incorporation following stimulation with anti-CD3 antibody, anti-T cell receptor antibody, anti-CD28 antibody, calcium ionophores, PMA, antigen presenting cells loaded with a peptide or protein antigen; B cell proliferation assays); measurement of the ability to kill or lyse other cells (such as cytotoxic T cell assays); measurements of the cytokines, chemokines, cell surface molecules, antibodies and other products of the cells (e.g., by flow cytometry, enzyme-linked immunosorbent assays, Western blot analysis, protein microarray analysis, immunoprecipitation analysis); measurement of biochemical markers of activation of immune cells or signaling pathways within immune cells (e.g., Western blot and immunoprecipitation analysis of tyrosine, serine or threonine phosphorylation, polypeptide cleavage, and formation or dissociation of protein complexes; protein array analysis; DNA transcriptional, profiling using DNA arrays or subtractive hybridization); measurements of cell death by apoptosis, necrosis, or other mechanisms (e.g., annexin V staining, TUNEL assays, gel electrophoresis to measure DNA laddering, histology; fluorogenic caspase assays, Western blot analysis of caspase substrates); measurement of the genes, proteins, and other molecules produced by immune cells (e.g., Northern blot analysis, polymerase chain reaction, DNA microarrays, protein microarrays, 2-dimensional gel electrophoresis, Western blot analysis, enzyme linked immunosorbent assays, flow cytometry); and measurement of clinical symptoms or outcomes such as improvement of autoimmune, neurodegenerative, and other diseases involving self proteins or self polypeptides (clinical scores, requirements for use of additional therapies, functional status, imaging studies) for example, by measuring relapse rate or disease severity (using clinical scores known to the ordinarily skilled artisan) in the case of multiple sclerosis, measuring blood glucose in the case of type I diabetes, or joint inflammation in the case of rheumatoid arthritis.

“Immune Modulatory Sequences (IMSs)” as used herein refers to compounds consisting of deoxynucleotides, ribonucleotides, or analogs thereof that modulate an autoimmune and/or inflammatory response. IMSs are typically oligonucleotides or a sequence of nucleotides incorporated in a vector (e.g., single-strand or double-stranded DNA, RNA, and/or oligonucleosides).

“Subjects” shall mean any animal, such as, for example, a human, non-human primate, horse, cow, dog, cat, mouse, rat, guinea pig or rabbit.

“Treating,” “treatment,” or “therapy” of a disease or disorder shall mean slowing, stopping or reversing the disease's progression, as evidenced by decreasing, cessation or elimination of either clinical or diagnostic symptoms, by administration of a polynucleotide encoding a self-polypeptide, either alone or in combination with another compound as described herein. “Treating,” “treatment,” or “therapy” also means a decrease in the severity of symptoms in an acute or chronic disease or disorder or a decrease in the relapse rate as for example in the case of a relapsing or remitting autoimmune disease course or a decrease in inflammation in the case of an inflammatory aspect of an autoimmune disease. In the preferred embodiment, treating a disease means reversing or stopping or mitigating the disease's progression, ideally to the point of eliminating the disease itself. As used herein, ameliorating a disease and treating a disease are equivalent.

“Preventing,” “prophylaxis,” or “prevention” of a disease or disorder as used in the context of this invention refers to the administration of a polynucleotide encoding a self-polypeptide, either alone or in combination with another compound as described herein, to prevent the occurrence or onset of a disease or disorder or some or all of the symptoms of a disease or disorder or to lessen the likelihood of the onset of a disease or disorder.

A “therapeutically or prophylactically effective amount” of a self-vector refers to an amount of the self-vector that is sufficient to treat or prevent the disease as, for example, by ameliorating or eliminating symptoms and/or the cause of the disease. For example, therapeutically effective amounts fall within broad range(s) and are determined through clinical trials and for a particular patient is determined based upon factors known to the skilled clinician, including, e.g., the severity of the disease, weight of the patient, age, and other factors.

In some embodiments, and as further described herein, NR2F6 is used to generate T cells that are tolerogenic. In one specific methodology this is accomplished using induced pluripotent stem cells (iPSC) generated from a Tn, a Tsm, and/or a T central memory cell can provide a virtually limitless source of long-lasting Tsm cells for adoptive therapy in chemotherapy treated patients with low Tsm frequencies. In some embodiments, and as further described herein, the Tsm cell used for adoptive therapy can be a CAR-Tsm cell. Without wishing to be bound by theory, it is believed that deriving an iPSC from a Tn, a T central memory cell, or a Tsm cell can retain the epigenetic landscape and developmental plasticity of the T cells in the iPSC and, in some embodiment, in a T cell differentiated from the iPSC. But Tsm cells mostly reside in the lymphoid tissues and very few of them are enter into peripheral blood stream (about 0.2-0.3%), making it challenging to obtain enough cells for reprogramming.

In some embodiments, the iPSC is preferably derived from a T cell of a T cell subset. For example, a T cell-derived iPSC (T-iPSC) can be derived from a naïve T (also referred to herein as a T naïve or a Tn) cell. A “Tn-iPSC” as used herein refers to an iPSC derived from a naïve T cell. In some embodiments, a T-iPSC can be derived from a stem memory T (T stem memory) cell. A “Tsm-iPSC” as used herein refers to an iPSC derived from a T stem memory cell. In contrast, a “CS-1-iPSC” as used herein refers to an iPSC derived from a human skin fibroblast cell. In some embodiments, a T cell, including a T cell of a T cell subset, can be separated from whole blood and/or a buffy coat using flow cytometry. In some embodiments, the T cell is a human cell. In some embodiments, the T cell can be derived from a subject in need of treatment. In some embodiments, the T cell is CD3+ and/or CD8+. In some embodiments, the T cell can further express at least one of CD45RA, CD62L, CD95, TCRαβ, CD4, CCR7, IL7Rα, CD27, and CD28. In some embodiments, a T naïve cell is defined as a cell that is CD8+CD62L+CD45RA+CCR7+CD95−CD45RO−. In some embodiments, a T stem memory cell is defined as a cell that is CD8+CD62L+CD45RA+CCR7+CD95+CD45RO−. In some embodiments, a T naïve cell and/or a T stem memory cell can also express CD27, CD28 and/or IL7Rα. In some embodiments, the T cell is a CD4+. In some embodiments, the T cell is a member of a T cell subset that has one or more features corresponding to a CD8+ T cell subset. In some embodiments generation of T regulatory cells is achieved by use of gene therapy is provided to induce an anti-oxidant environment prior to stem cell administration to stimulate T regulatory cell generation. In one embodiment gene therapy with superoxide dismutase is disclosed in order to modulate the microenvironment. The gene therapy can be administered in a higher dose to provide a systemic protective effect to stem cell compartments. One benefit of the systemic effect is that a dose of gene therapy can be administered to a patient and provide the desired effect at a variety of locations. This alleviates the need to locate all locations in need of treatment. The gene therapy can be administered via intravenous administration, intra-bone marrow administration, intra-arterial administration, intra-cardiac injection, intracerebral injection, intraspinal injection, intra-peritoneal injection, intra-muscular injection, subcutaneous injection, parenteral administration, intra-rectal administration, intra-tracheal injection, intra-nasal administration, intradermal injection, and the like. Administration of these compositions can be via any common route so long as the target tissue is available via that route. The vector for delivering superoxide dismutase may be any vector that may conveniently be subjected to recombinant DNA procedures, and the choice of vector will often depend on the host cell into which it is to be introduced. Thus, the vector may be an autonomously replicating vector, i.e. a vector that exists as an extrachromosomal entity, the replication of which is independent of chromosomal replication; examples of such a vector are a plasmid, phage, cosmid, mini-chromosome or virus. Alternatively, the vector may be one which, when introduced in a host cell, is integrated in the host cell genome and replicated together with the chromosome(s) into which it has been integrated. Additionally, the gene therapy can be administered in a higher dose to provide a systemic protective effect. The benefit of the systemic effect is that a dose of gene therapy can be administered to a patient and provide the desired effect at any necessary locations. This alleviates the need to locate all locations in need of treatment.

The T regulatory cell inducing gene therapy of the invention can be administered to the human or other animal after inflammation induced stem cell damage such as in osteoarthritis in an amount that is effective for diminishing damage to the respiratory, gastrointestinal and the hematopoietic systems after sublethal irradiation or for increasing the survival rate after lethal inflammation. The gene therapy may also be effective when administered prior to or during exposure to inflammation. Another dosing regimen would include multiple doses given both prior and/or following the exposure to inflammation. Those of skill in the art are well aware of how to apply adenoviral delivery to in vivo and ex vivo situations. For viral vectors, one generally will prepare a viral vector stock. Depending on the kind of virus and the titer attainable, one will deliver 1 to 10, 10 to 50, 100-1000, or up to 1.times.10.sup.4, 1.times.10.sup.5, 1.times.10.sup.6, 1.times.10.sup.7, 1.times.10.sup.8, 1.times.10.sup.9, 1.times.10.sup.10, 1.times.10.sup.11, or 1.times.10.sup.12 infectious particles to the patient in a pharmaceutically acceptable composition as discussed below. Various routes are contemplated for osteoarthritis. Where discrete locations or tissues may be identified, a variety of direct, local and regional approaches may be taken. For example, an organ may be directly injected with the adenovirus. The adenovirus can be delivered by a catheter having access to the tissue. One may utilize the local vasculature to introduce the vector into the tissue or organ by injecting a supporting vein or artery. A more distal blood supply route also may be utilized. It may also be beneficial to treat the surrounding tissue, not just the affected tissue. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals. Appropriate salts and buffers can be used to render delivery vectors stable and allow for uptake by target cells. Aqueous compositions of the gene therapy can include an effective amount of the vectors, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the gene therapy, its use in therapeutic compositions is contemplated. Supplementary active ingredients also can be incorporated into the compositions.

The cells of the invention comprising NR2F6 overexpressing T cells may be used to treat various forms of autoimmune disease. A non-exclusive description of some of the major types of autoimmune disease is provided below.

Multiple Sclerosis. Multiple sclerosis (MS) is the most common demyelinating disorder of the CNS and affects 350,000 Americans and one million people worldwide. Onset of symptoms typically occurs between 20 and 40 years of age and manifests as an acute or sub-acute attack of unilateral visual impairment, muscle weakness, paresthesias, ataxia, vertigo, urinary incontinence, dysarthria, or mental disturbance (in order of decreasing frequency). Such symptoms result from focal lesions of demyelination which cause both negative conduction abnormalities due to slowed axonal conduction, and positive conduction abnormalities due to ectopic impulse generation (e.g., Lhermitte's symptom). Diagnosis of MS is based upon a history including at least two distinct attacks of neurologic dysfunction that are separated in time, produce objective clinical evidence of neurologic dysfunction, and involve separate areas of the CNS white matter. Laboratory studies providing additional objective evidence supporting the diagnosis of MS include magnetic resonance imaging (MRI) of CNS white matter lesions, cerebral spinal fluid (CSF) oligoclonal banding of IgG, and abnormal evoked responses. Although most patients experience a gradually progressive relapsing remitting disease course, the clinical course of MS varies greatly between individuals and can range from being limited to several mild attacks over a lifetime to fulminant chronic progressive disease. A quantitative increase in myelin-autoreactive T cells with the capacity to secrete IFN-gamma is associated with the pathogenesis of MS and EAE.

The autoantigen targets of the autoimmune response in autoimmune demyelinating diseases, such as multiple sclerosis and experimental autoimmune encephalomyelitis (EAE), may comprise epitopes from proteolipid protein (PLP); myelin basic protein (MBP); myelin oligodendrocyte glycoprotein (MOG); cyclic nucleotide phosphodiesterase (CNPase); myelin-associated glycoprotein (MAG), and myelin-associated oligodendrocytic basic protein (MBOP); alpha-B-crystallin (a heat shock protein); viral and bacterial mimicry peptides, e.g., influenza, herpes viruses, hepatitis B virus, etc.; OSP (oligodendrocyte specific-protein); citrulline-modified MBP (the C8 isoform of MBP in which 6 arginines have been de-imminated to citrulline), etc. The integral membrane protein PLP is a dominant autoantigen of myelin. Determinants of PLP antigenicity have been identified in several mouse strains, and include residues 139-151, 103-116, 215-232, 43-64 and 178-191. At least 26 MBP epitopes have been reported (Meinl et al., J Clin Invest 92, 2633-43, 1993). Notable are residues 1-11, 59-76 and 87-99. Immunodominant MOG epitopes that have been identified in several mouse strains include residues 1-22, 35-55, 64-96.

In human MS patients the following myelin proteins and epitopes were identified as targets of the autoimmune T and B cell response. Antibody eluted from MS brain plaques recognized myelin basic protein (MBP) peptide 83-97 (Wucherpfennig et al., J Clin Invest 100:1114-1122, 1997). Another study found approximately 50% of MS patients having peripheral blood lymphocyte (PBL) T cell reactivity against myelin oligodendrocyte glycoprotein (MOG) (6-10% control), 20% reactive against MBP (8-12% control), 8% reactive against PLP (0% control), 0% reactive MAG (0% control). In this study 7 of 10 MOG reactive patients had T cell proliferative responses focused on one of 3 peptide epitopes, including MOG 1-22, MOG 34-56, MOG 64-96 (Kerlero de Rosbo et al., Eur J Immunol 27, 3059-69, 1997). T and B cell (brain lesion-eluted Ab) response focused on MBP 87-99 (Oksenberg et al., Nature 362, 68-70, 1993). In MBP 87-99, the amino acid motif HFFK is a dominant target of both the T and B cell response (Wucherpfennig et al., J Clin Invest 100, 1114-22, 1997). Another study observed lymphocyte reactivity against myelin-associated oligodendrocytic basic protein (MOBP), including residues MOBP 21-39 and MOBP 37-60 (Holz et al., J Immunol 164, 1103-9, 2000). Using immunogold conjugates of MOG and MBP peptides to stain MS and control brains both MBP and MOG peptides were recognized by MS plaque-bound Abs (Genain and Hauser, Methods 10, 420-34, 1996).

Rheumatoid Arthritis. Rheumatoid arthritis (RA) is a chronic autoimmune inflammatory synovitis affecting 0.8% of the world population. It is characterized by chronic inflammatory synovitis that causes erosive joint destruction. RA is mediated by T cells, B cells and macrophages.

Evidence that T cells play a critical role in RA includes the (1) predominance of CD4+. T cells infiltrating the synovium, (2) clinical improvement associated with suppression of T cell function with drugs such as cyclosporine, and (3) the association of RA with certain HLA-DR alleles. The HLA-DR alleles associated with RA contain a similar sequence of amino acids at positions 67-74 in the third hypervariable region of the R chain that are involved in peptide binding and presentation to T cells. RA is mediated by autoreactive T cells that recognize a self-protein, or modified self-protein, present in synovial joints. Autoantigens that are targeted in RA comprise, e.g., epitopes from type II collagen; hnRNP; A2/RA33; Sa; filaggrin; keratin; citrulline; cartilage proteins including gp39; collagens type I, III, IV, V, IX, XI; HSP-65/60; IgM (rheumatoid factor); RNA polymerase; hnRNP-B1; hnRNP-D; cardiolipin; aldolase A; citrulline-modified filaggrin and fibrin. Autoantibodies that recognize filaggrin peptides containing a modified arginine residue (de-iminated to form citrulline) have been identified in the serum of a high proportion of RA patients. Autoreactive T and B cell responses are both directed against the same immunodominant type II collagen (CII) peptide 257-270 in some patients.

Insulin Dependent Diabetes Mellitus. Human type I or insulin-dependent diabetes mellitus (IDDM) is characterized by autoimmune destruction of the R cells in the pancreatic islets of Langerhans. The depletion of β cells results in an inability to regulate levels of glucose in the blood. Overt diabetes occurs when the level of glucose in the blood rises above a specific level, usually about 250 mg/dl. In humans a long presymptomatic period precedes the onset of diabetes. During this period there is a gradual loss of pancreatic beta cell function. The development of disease is implicated by the presence of autoantibodies against insulin, glutamic acid decarboxylase, and the tyrosine phosphatase IA2 (IA2).

Markers that may be evaluated during the presymptomatic stage are the presence of insulitis in the pancreas, the level and frequency of islet cell antibodies, islet cell surface antibodies, aberrant expression of Class II MHC molecules on pancreatic beta cells, glucose concentration in the blood, and the plasma concentration of insulin. An increase in the number of T lymphocytes in the pancreas, islet cell antibodies and blood glucose is indicative of the disease, as is a decrease in insulin concentration.

The Non-Obese Diabetic (NOD) mouse is an animal model with many clinical, immunological, and histopathological features in common with human IDDM. NOD mice spontaneously develop inflammation of the islets and destruction of the β cells, which leads to hyperglycemia and overt diabetes. Both CD4+ and CD8+ T cells are required for diabetes to develop, although the roles of each remain unclear. It has been shown that administration of insulin or GAD, as proteins, under tolerizing conditions to NOD mice prevents disease and down-regulates responses to the other autoantigens.

The presence of combinations of autoantibodies with various specificities in serum are highly sensitive and specific for human type I diabetes mellitus. For example, the presence of autoantibodies against GAD and/or IA-2 is approximately 98% sensitive and 99% specific for identifying type I diabetes mellitus from control serum. In non-diabetic first degree relatives of type I diabetes patients, the presence of autoantibodies specific for two of the three autoantigens including GAD, insulin and IA-2 conveys a positive predictive value of >90% for development of type IDM within 5 years.

Autoantigens targeted in human insulin dependent diabetes mellitus may include, for example, tyrosine phosphatase IA-2; IA-2β; glutamic acid decarboxylase (GAD) both the 65 kDa and 67 kDa forms; carboxypeptidase H; insulin; proinsulin; heat shock proteins (HSP); glima 38; islet cell antigen 69 KDa (ICA69); p52; two ganglioside antigens (GT3 and GM2-1); islet-specific glucose-6-phosphatase-related protein (IGRP); and an islet cell glucose transporter (GLUT 2).

Human IDDM is currently treated by monitoring blood glucose levels to guide injection, or pump-based delivery, of recombinant insulin. Diet and exercise regimens contribute to achieving adequate blood glucose control.

Autoimmune Uveitis. Autoimmune uveitis is an autoimmune disease of the eye that is estimated to affect 400,000 people, with an incidence of 43,000 new cases per year in the U.S. Autoimmune uveitis is currently treated with steroids, immunosuppressive agents such as methotrexate and cyclosporin, intravenous immunoglobulin, and TNFα-antagonists.

Experimental autoimmune uveitis (EAU) is a T cell-mediated autoimmune disease that targets neural retina, uvea, and related tissues in the eye. EAU shares many clinical and immunological features with human autoimmune uveitis, and is induced by peripheral administration of uveitogenic peptide emulsified in Complete Freund's Adjuvant (CFA).

Autoantigens targeted by the autoimmune response in human autoimmune uveitis may include S-antigen, interphotoreceptor retinoid binding protein (IRBP), rhodopsin, and recoverin.

Primary Billiary Cirrhosis. Primary Biliary Cirrhosis (PBC) is an organ-specific autoimmune disease that predominantly affects women between 40-60 years of age. The prevalence reported among this group approaches 1 per 1,000. PBC is characterized by progressive destruction of intrahepatic biliary epithelial cells (IBEC) lining the small intrahepatic bile ducts. This leads to obstruction and interference with bile secretion, causing eventual cirrhosis. Association with other autoimmune diseases characterized by epithelium lining/secretory system damage has been reported, including Sjögren's Syndrome, CREST Syndrome, Autoimmune Thyroid Disease and Rheumatoid Arthritis. Attention regarding the driving antigen(s) has focused on the mitochondria for over 50 years, leading to the discovery of the antimitochondrial antibody (AMA) (Gershwin et al., Immunol Rev 174:210-225, 2000); (Mackay et al., Immunol Rev 174:226-237, 2000). AMA soon became a cornerstone for laboratory diagnosis of PBC, present in serum of 90-95% patients long before clinical symptoms appear. Autoantigenic reactivities in the mitochondria were designated as M1 and M2. M2 reactivity is directed against a family of components of 48-74 kDa. M2 represents multiple autoantigenic subunits of enzymes of the 2-oxoacid dehydrogenase complex (2-OADC) and is another example of the self-protein, -polypeptide, or -peptide of the instant invention. Studies identifying the role of pyruvate dehydrogenase complex (PDC) antigens in the etiopathogenesis of PBC support the concept that PDC plays a central role in the induction of the disease (Gershwin et al., Immunol Rev 174:210-225, 2000); (Mackay et al., Immunol Rev 174:226-237, 2000). The most frequent reactivity in 95% of cases of PBC is the E2 74 kDa subunit, belonging to the PDC-E2. There exist related but distinct complexes including: 2-oxoglutarate dehydrogenase complex (OGDC) and branched-chain (BC) 2-OADC. Three constituent enzymes (E1, 2, 3) contribute to the catalytic function which is to transform the 2-oxoacid substrate to acyl co-enzyme A (CoA), with reduction of NAD+ to NADH. Mammalian PDC contains—an additional component, termed protein X or E-3 Binding protein: (E3BP). In PBC patients, the, major antigenic response is directed against PDC-E2 and E3BP. The E2 polypeptide contains two tandemly repeated lipoyl domains, while E3BP has a single lipoyl domain. The lipoyl domain is found in a number of autoantigen targets of PBC and is referred to herein as the “PBC lipoyl domain.” PBC is treated with glucocorticoids and immunosuppressive agents including methotrexate and cyclosporin A.

A murine model of experimental autoimmune cholangitis (EAC) uses intraperitoneal (i.p.) sensitization with mammalian PDC in female SJL/J mice, inducing non-suppurative destructive cholangitis (NSDC) and production of AMA (Jones, J Clin Pathol 53:813-21, 2000).

Other Autoimmune Diseases And Associated Autoantigens. Autoantigens for myasthenia gravis may include epitopes within the acetylcholine receptor. Autoantigens targeted in pemphigus vulgaris may include desmoglein-3. Sjogren's syndrome antigens may include SSA (Ro); SSB (La); and fodrin. The dominant autoantigen for pemphigus vulgaris may include desmoglein-3. Panels for myositis may include tRNA synthetases (e.g., threonyl, histidyl, alanyl, isoleucyl, and glycyl); Ku; Scl; SSA; U1 Sn ribonuclear protein; Mi-1; Mi-1; Jo-1; Ku; and SRP. Panels for scleroderma may include Scl-70; centromere; U1 ribonuclear proteins; and fibrillarin. Panels for pernicious anemia may include intrinsic factor; and glycoprotein beta subunit of gastric H/K ATPase. Epitope Antigens for systemic lupus erythematosus (SLE) may include DNA; phospholipids; nuclear antigens; Ro; La; U1 ribonucleoprotein; Ro60 (SS-A); Ro52 (SS-A); La (SS-B); calreticulin; Grp78; Scl-70; histone; Sm protein; and chromatin, etc. For Grave's disease epitopes may include the Na+/I− symporter; thyrotropin receptor; Tg; and TPO.

Graft Versus Host Disease. One of the greatest limitations of tissue and organ transplantation in humans is rejection of the tissue transplant by the recipient's immune system. It is well established that the greater the matching of the MHC class I and II (HLA-A, HLA-B, and HLA-DR) alleles between donor and recipient the better the graft survival. Graft versus host disease (GVHD) causes significant morbidity and mortality in patients receiving transplants containing allogeneic hematopoietic cells. Hematopoietic cells are present in bone-marrow transplants, stem cell transplants, and other transplants. Approximately 50% of patients receiving a transplant from a HLA-matched sibling will develop moderate to severe GVHD, and the incidence is much higher in non-HLA-matched grafts. One-third of patients that develop moderate to severe GVHD will die as a result. T lymphocytes and other immune cell in the donor graft attack the recipients' cells that express polypeptides variations in their amino acid sequences, particularly variations in proteins encoded in the major histocompatibility complex (MHC) gene complex on chromosome 6 in humans. The most influential proteins for GVHD in transplants involving allogeneic hematopoietic cells are the highly polymorphic (extensive amino acid variation between people) class I proteins (HLA-A, —B, and -C) and the class II proteins (DRB1, DQB1, and DPB1) (Appelbaum, Nature 411:385-389, 2001). Even when the MHC class I alleles are serologically ‘matched’ between donor and recipient, DNA sequencing reveals there are allele-level mismatches in 30% of cases providing a basis for class I-directed GVHD even in matched donor-recipient pairs (Appelbaum, Nature 411, 385-389, 2001). The minor histocompatibility self-antigens GVHD frequently causes damage to the skin, intestine, liver, lung, and pancreas. GVHD is treated with glucocorticoids, cyclosporine, methotrexate, fludarabine, and OKT3.

Tissue Transplant Rejection. Immune rejection of tissue transplants, including lung, heart, liver, kidney, pancreas, and other organs and tissues, is mediated by immune responses in the transplant recipient directed against the transplanted organ. Allogeneic transplanted organs contain proteins with variations in their amino acid sequences when compared to the amino acid sequences of the transplant recipient. Because the amino acid sequences of the transplanted organ differ from those of the transplant recipient they frequently elicit an immune response in the recipient against the transplanted organ. Rejection of transplanted organs is a major complication and limitation of tissue transplant, and can cause failure of the transplanted organ in the recipient. The chronic inflammation that results from rejection frequently leads to dysfunction in the transplanted organ. Transplant recipients are currently treated with a variety of immunosuppressive agents to prevent and suppress rejection. These agents include glucocorticoids, cyclosporin A, Cellcept, FK-506, and OKT3.

One skilled in the art will appreciate that these methods, compositions, and cells are and may be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. It will be apparent to one skilled in the art that varying substitutions and modifications may be made to the invention disclosed herein without departing from the scope and spirit of the invention. Those skilled in the art recognize that the aspects and embodiments of the invention set forth herein may be practiced separate from each other or in conjunction with each other. Therefore, combinations of separate embodiments are within the scope of the invention as disclosed herein. All patents and publications mentioned in the specification are indicative of the levels of those skilled in the art to which the invention pertains. All patents and publications are herein incorporated by reference to the same extent as if each individual publication was specifically and individually indicated to be incorporated by reference. According to one embodiment of the present invention, the chimeric antigen receptor may include two intracellular signaling domains. For example, the chimeric antigen receptor may include a first intracellular signaling domain linked to the transmembrane domain and a second intracellular signaling domain linked to a terminal of the first intracellular signaling domain that is not linked with the transmembrane domain. According to a more specific embodiment, the first intracellular signaling domain may include the whole or a portion of any one selected from the group consisting of OX40 (CD134), OX40 ligand (OX40L, CD252), 4-1BB (CD137), CD28, DAP10, CD3-zeta (CD3) and DAP12, and the second intracellular signaling domain may include the whole or a portion of any one selected from the group consisting of OX40 ligand, CD3-zeta and DAP12. In this case, at least one of the first intracellular signaling domain and the second intracellular signaling domain includes the whole or a portion of OX40 ligand. For example, the chimeric antigen receptor may include a first intracellular signaling domain containing the whole or a portion of OX40 ligand and a second intracellular signaling domain containing the whole or a portion of any one selected from CD3-zeta and DAP12. Further, for example, the chimeric antigen receptor may include a first intracellular signaling domain containing the whole or a portion of any one selected from the group consisting of CD3-zeta and DAP12 and a second intracellular signaling domain containing the whole or a portion of OX40 ligand. According to another embodiment of the present invention, the chimeric antigen receptor may include three intracellular signaling domains. For example, the chimeric antigen receptor may include: a tirst intracellular signaling domain linked to the transmembrane domain; a second intracellular signaling domain linked to a terminal of the first intracellular signaling domain that is not linked with the transmembrane domain; and a third intracellular signaling domain linked to a terminal of the second intracellular signaling domain that is not linked with the first intracellular signaling domain. According to a more specific embodiment, the first intracellular signaling domain may include the whole or a portion of any one selected from the group consisting of 4-1BB, OX40, OX40 ligand, CD28 and DAP10, the second intracellular signaling domain may include the whole or a portion of any one selected from the group consisting of OX40 ligand, OX40 and 4-1BB, and the third intracellular signaling domain may include the whole or a portion of any one selected from the group consisting of OX40 ligand, CD3-zeta and DAP12. In such a case, at least one of the first intracellular signaling domain, the second intracellular signaling domain and the third intracellular signaling domain may include the whole or a portion of OX40 ligand. In another aspect, the present invention may provide a chimeric antigen receptor, which includes: a first intracellular signaling domain containing the whole or a portion of any one selected from the group consisting of CD28 and 4-1BB; a second intracellular signaling domain containing the whole or a portion of any one selected from the group consisting of OX40 ligand, OX40 and 4-1BB; and a third intracellular signaling domain containing the whole or a portion of CD3-zeta, wherein the first, second and third intracellular signaling domains are arranged in order from the cell membrane toward the inside of the cell. According to one embodiment of the present invention, the above respective domains may be directly linked to one another or may be linked by a linker. According to one embodiment of the present invention, the chimeric antigen receptor may further include: a transmembrane domain linked to the first intracellular signaling domain; a spacer domain linked to the transmembrane domain; and an extracellular domain linked to the spacer domain. In addition, the chimeric antigen receptor may further include a signal sequence linked to the extracellular domain. According to one embodiment of the present invention, the above respective domains may be directly linked to one another or may be linked by a linker.

According to one embodiment of the present invention, the extracellular domain is a domain for specifically binding with an antibody or specifically recognizing an antigen, for example, an Fc receptor, an antigen-binding fragment of an antibody such as a single-chain variable fragment (ScFv), NK receptor (natural cytotoxicity receptor), NKG2D, 2B4 or DNAM-1, etc. Thus, in the present disclosure, the term “extracellular domain” is used with the same meanings as the “antigenic recognition site”, “antigen-binding fragment” and/or “antibody binding site.” The chimeric antigen receptor according to an embodiment of the present invention may include an Fc receptor as the extracellular domain, and therefore, can be used along with a variety of antibodies depending on cell types of cancer to be treated. According to one embodiment, the Fc receptor may include any one selected from the group consisting of CD16, CD32, CD64, CD23 and CD89, and variants thereof. According to a more specific embodiment, the Fc receptor may include CD16 or variants thereof, and most specifically, may include the whole or a portion of CD16 V158 variant (CD16V). According to another embodiment, the chimeric antigen receptor of the present invention may include, as the extracellular domain, an antigen-binding fragment of an antibody which directly recognizes the antigen without co-administration along with the antibody. According to one embodiment, the antigen-binding fragment may be an Fab fragment, F(ab′) fragment, F(ab′)2 fragment or Fv fragment. According to one embodiment of the present invention, the antibody may be any one of various types of antibodies capable of binding antigen-specifically. For example, the antibody may be one in which one light chain and one heavy chain are bonded with each other, or one in which two light chains and two heavy chains are bonded with each other. For example, when two light chains and two heavy chains are bonded with each other, the antibody may be one in which the first unit including the first light chain and the first heavy chain bonded with each other and the second unit including the second light chain and the second heavy chain bonded with each other are combined with each other. The bond may be a disulfide bond, but it is not limited thereto. According to an embodiment of the present invention, the above two units may be the same as or different from each other. For example, the first unit including the first light chain and the first heavy chain and the second unit including the second light chain and the second heavy chain may be the same as or different from each other. As such, an antibody prepared to recognize two different antigens by the first unit and the second unit, respectively, is commonly referred to as a ‘bispecific antibody’ in the related art. In addition, for example, the antibody may be one in which the above three or more units are combined with one another. The antigen-binding fragment of the present invention may be derived from various types of antibodies as described above, but it is not limited thereto. According to another embodiment of the present invention, the extracellular domain used herein may be a NK receptor (natural cytotoxicity receptor). According to a specific embodiment, the NK receptor may include NKp46, NKp30, NKp44, NKp80 and NKp65 receptors, but it is not limited thereto. According to one embodiment, the signal sequence may include the whole or a portion of CD16. According to another embodiment, the extracellular domain may include the whole or a portion of CD16 V158 variant (CD16V). According to another embodiment, the spacer domain may include the whole or a portion of any one selected from the group consisting of CD8.alpha. (CD8-alpha) and CD28. According to another embodiment, the transmembrane domain may include the whole or a portion of any one selected from the group consisting of CD8.alpha. and CD28.

1. A tolerance promoting T cell generated through augmentation of NR2F6 levels in a cell selected from the group consisting of: a T cell, a T cell progenitor, and a pluripotent stem cell giving rise to a tolerance promoting T cell.

2. The tolerance promoting T cell of claim 1, wherein augmentation of NR2F6 is achieved through introduction of one or more short hairpin RNA molecules targeting NR2F6.

3. The tolerance promoting T cell of claim 2, wherein said introduction of one or more short hairpin RNA molecules targeting NR2F6 results in upregulation of NR2F6 activity.

4. The tolerance promoting T cell of claim 1, wherein said tolerance promoting T cell is a chimeric antigen receptor (CAR) T cell.

5. The tolerance promoting T cell of claim 4, wherein said chimeric antigen receptor recognizes an autoantigen selected from the group consisting of: GAD65, insulin, insulin beta chain, IA2, phogrin, hsp90b, carboxypeptidase E, islet glucose 6 phosphatase-related protein, islet amyloid polypeptide, Reg3a, ICA69, imogen 38, peripherin, sox13, GAD67, Hsp65, DNAJ, immunoglobulin binding protein, heterogeneous nuclear ribonucleoprotein A2, calpastatin, type II collagen, glucose-6-phosphate isomerase, gp39, mannose binding lectin, citrullinated vimentin, fibrinogen, alpha enolase, myelin basic protein, anoctamin-2, myelin oligodendrocyte glycoprotein, KIR4.1, and aquaporin-4, CRYAB, PLP.

6. The tolerance promoting T cell of claim 1, wherein said tolerance promoting T cell inhibits maturation of dendritic cells.

7. The tolerance promoting T cell of claim 6, wherein said dendritic cell maturation is inhibited by a method selected from the group consisting of: IL-12 production, IL-15 production, IL-18 production, IL-17 production, IL-21 production, IL-23 production, IL-27 production, IL-33 production, IL-1 beta production, HMGB1 production, interferon alpha production, lymphotoxin production, TNF-alpha production, MIP-1 alpha production, MIP-1 beta production, RANTES production, TRANCE production, MCP production, expression of CD40, expression of CD80, expression of CD86, expression of LFA-1, expression of ICAM-1, transporter associated protein-1, antigen presentation activity.

8. The tolerance promoting T cell of claim 1, wherein said T cell is capable of suppressing proliferation of other T cells.

9. A method of generating a tolerance promoting T cell comprising augmenting NR2F6 levels in a cell selected from the group consisting of: a T cell, a T cell progenitor, and a pluripotent stem cell.

10. The method of claim 9, wherein augmentation of NR2F6 is achieved through introduction of one or more short hairpin RNA molecules targeting NR2F6.

11. The method of claim 10, wherein said introduction of one or more short hairpin RNA molecules targeting NR2F6 results in upregulation of NR2F6 activity.

12. The method of claim 1, wherein said tolerance promoting T cell is a chimeric antigen receptor (CAR) T cell.

13. The method of claim 12, wherein said chimeric antigen receptor recognizes an autoantigen selected from the group consisting of: GAD65, insulin, insulin beta chain, IA2, phogrin, hsp90b, carboxypeptidase E, islet glucose 6 phosphatase-related protein, islet amyloid polypeptide, Reg3a, ICA69, imogen 38, peripherin, sox13, GAD67, Hsp65, DNAJ, immunoglobulin binding protein, heterogeneous nuclear ribonucleoprotein A2, calpastatin, type II collagen, glucose-6-phosphate isomerase, gp39, mannose binding lectin, citrullinated vimentin, fibrinogen, alpha enolase, myelin basic protein, anoctamin-2, myelin oligodendrocyte glycoprotein, KIR4.1, and aquaporin-4, CRYAB, PLP.

14. The method of claim 9, wherein said tolerance promoting T cell inhibits maturation of dendritic cells.

15. The method of claim 14, wherein said dendritic cell maturation is inhibited by a method selected from the group consisting of: IL-12 production, IL-15 production, IL-18 production, IL-17 production, IL-21 production, IL-23 production, IL-27 production, IL-33 production, IL-1 beta production, HMGB1 production, interferon alpha production, lymphotoxin production, TNF-alpha production, MIP-1 alpha production, MIP-1 beta production, RANTES production, TRANCE production, MCP production, expression of CD40, expression of CD80, expression of CD86, expression of LFA-1, expression of ICAM-1, transporter associated protein-1, antigen presentation activity.

16. The method of claim 9, wherein said T cell is capable of suppressing proliferation of other T cells.
